(19) United States
(12) Patent Application Publication
(10) Pub. No.: US 2025/0387281 A1
(43) Pub. Date: Dec. 25, 2025

(54) DECOUPLED MEASURING SYSTEM FOR VERTICAL POSITION CHANGES

(71) Applicant: Siemens Healthineers AG, Forchheim (DE)

(72) Inventor: Wolfgang NEUBER, Eschenbach (DE)

(73) Assignee: Siemens Healthineers AG, Forchheim (DE)

(21) Appl. No.: 19/247,484

(22) Filed: Jun. 24, 2025

(30) Foreign Application Priority Data
Jun. 25, 2024 (DE) 10 2024 205 894.7

(51) Int. Cl. A61G 13/06; A61G 13/10

(52) U.S. Cl. CPC A61G 13/06; A61G 13/10

(57) ABSTRACT

A system for ascertaining position information for a measuring position of a measurement object, comprises: a sensor tape; a sensor configured to capture position information on the sensor tape; a traction cable configured to transmit the measuring position of the measurement object to the sensor; and at least one deflection pulley configured to deflect the traction cable from a first direction in a second direction.

### CROSS-REFERENCE TO RELATED APPLICATION(S)

The present application claims priority under 35 U.S.C. § 119 to German Patent Application No. 10 2024 205 894.7, filed Jun. 25, 2024, the entire contents of which is incorporated herein by reference.

### FIELD

One or more example embodiments of the present invention relate to a measuring system for ascertaining position information for a measuring position of a measurement object, a table with such a measuring system and a medical apparatus with such a table and/or such a measuring system.

### BACKGROUND

On the movement of machine axes, for example in medical devices, it is advantageous to be able to capture or measure the respective paths of the axes or corresponding target positions exactly. Usually, such measuring tasks are performed using linear measuring systems, which are arranged directly at/on the axis to be measured and enable direct and continuous measurement of the position of the measurement object. Herein, linear measuring systems are in particular characterized by their high measuring accuracy and robustness. Alternatively, cable pull transducers can be used if the use of linear measuring systems is not possible. In addition, indirect measurements on the drive kinematics with subsequent calculation of the resulting drive position/movement can be used.

However, these alternative measuring methods have various disadvantages. With indirect measurement, there is no discrete measurement and so geometric errors of the kinematics can influence the result. Cable pull transducers have measuring inaccuracies, primarily due to the cable drum principle, i.e. the winding and unwinding of a cable or thread on a spool over a plurality of revolutions or even a plurality of layers with a corresponding number of revolutions. In addition, the inaccuracies in the position measurement with cable pull transducers are not constant over the entire measuring range. Even correction values, such as, for example, gradient compensation, cannot ensure sufficiently good precision for specific requirements, such as, for example, position capturing in radiotherapy applications. The greater the measuring length of the cable pull transducer, the greater the measuring inaccuracy. Therefore, cable pull transducers are unsuitable for specific applications or significantly less suitable than linear measuring systems.

For some applications where precise position measurement is required, linear measuring systems can be arranged on or at the axes or measuring positions to be measured. For example, in the case of scissor lifting modules, in particular when used in radiotherapy systems where high precision is required, the vertical position/vertical travel cannot be measured by a linear measuring system.

### SUMMARY

An object of one or more example embodiments of the present invention is to enable a reliable, exact measurement of measuring positions, in particular in the height direction (y-direction), for apparatuses in which known measuring systems, in particular linear measuring systems, cannot be used due to the geometric nature of the apparatus.

A measuring system for ascertaining position information for a measuring position of a measurement object is proposed. The measuring system comprises a sensor tape and a sensor that captures position information on the sensor tape. In addition, the measuring system comprises a traction cable for transmitting a measuring position of the measurement object to the sensor and a deflection pulley that deflects the traction cable from a first direction into a second direction.

Preferably, the position information can describe the (ideally actual) spatial location of the measuring position of the measurement object. Preferably, the position information can comprise a three-dimensional and/or two-dimensional description (in x and y) of the measurement point based on a reference object, in particular a reference point, via a coordinate system. Herein, the reference point (e.g. zero point of the coordinate system) is preferably arranged on or at the measuring system, in particular the sensor tape. In particular, the reference point can be the starting point of the sensor and/or the point at which the sensor is arranged in the starting position of the measurement object on the sensor tape. Alternatively, the reference point (e.g. zero point of the coordinate system) can preferably be arranged on or at the measurement object. The measuring position can preferably be the position on the measurement object at which the traction cable is connected to the measurement object. Alternatively, the measurement point can in particular also be another point at and/or on the measurement object, for example the geometric center point of the measurement object or a center point of a surface of the measurement object. In this case, the location/position of the referenced point on/in the measurement object is preferably known. The position information can in particular be a distance and/or a path length. In particular, the position information can be a height specification and/or a length value of a distance in the y-direction of a coordinate system. In particular, the position information can be read directly from the measuring system as a length value and/or ascertained by the sensor on the position tape and transferred/output to a system interface and/or user interface.

The measurement object is in particular the object on which the measurement point is arranged. The measurement object can preferably comprise the measuring system. Alternatively, the measuring system can be part of another object/apparatus or be arranged outside the measurement object. The measurement object can in particular be a medical apparatus. For example, the measurement object can be a medical imaging apparatus, a medical therapy apparatus and/or a part/component of a medical apparatus. For example, the measurement object can be a patient bench.

The sensor tape can in particular be a scale and/or a measuring tape. The sensor tape can in particular be embodied as a tape and/or a rod, preferably made of metal, ceramic, glass or glass-ceramic. However, alternatively, the sensor tape can also have another geometric shape on which a sensor can be arranged for a measurement. The sensor tape can in particular have a smooth surface. Preferably, the sensor tape can comprise a groove, recess, strip or guide with which the sensor can preferably be positioned and/or guided on the sensor tape. The sensor tape can preferably have a coding, scale or measurement. For example, a special code pattern on the sensor tape can enable precise position determination by the sensor without knowledge of the previous position. Calibration or zeroing is advantageously not necessary in this case. Preferably, in the case of sensors with an incremental output signal, the sensor can comprise a calibration mark, reference position, reference magnet and/or index pulse that enables calibration and/or the determination of the absolute position. For example, the sensor tape can comprise a magnetic strip. In this case, the magnetic strip can preferably comprise north poles and/or south poles alternating at specified intervals.

The sensor can also be referred to as a sensor head, read head, scanner and/or displacement transducer. The sensor can preferably be a displacement sensor. The sensor can preferably be a magnetic, inductive, magnetostrictive or scanning sensor. For example, the sensor can comprise a magnetoresistive sensor or Hall sensor. The sensor can preferably convert a linear movement into analog or digital signals, in particular position information. The sensor can in particular be arranged on the sensor tape and/or surround it. Preferably, the sensor can only be moved linearly in two directions along the sensor tape. In other words, the sensor has only one degree of freedom of movement, for example in the x-direction in a coordinate system arranged at the origin of a linear sensor tape. In particular, the sensor can be an absolute measuring sensor that outputs an absolute position on a measuring path, in particular the sensor tape, as an output signal. Alternatively, the sensor can return an incremental output signal. In this case, it is preferably necessary to determine the absolute position by zeroing and/or calibrating. The sensor can preferably be arranged in a movable housing. The sensor with the sensor tape can be referred to as a length measuring system, linear measuring apparatus and/or tape sensor. Preferably, the sensor can be guided and/or measure in a contactless manner over/on the sensor tape so that the measurement can take place without friction and wear.

The traction cable can in particular refer to a cable under tensile stress. The traction cable can preferably comprise metal and/or plastic. In particular, the traction cable can comprise a steel cable, a plastic cable, a carbon fiber cable and/or a cable made of a material with a high modulus of elasticity, preferably 200 GPa or greater. The traction cable preferably has a low, quantifiable and continuous linear expansion when a specific tensile force and/or restoring force is applied. Preferably, the traction cable is temperature-resistant, in particular so that the traction cable exhibits the smallest change in length when the temperature changes. The traction cable is in particular arranged between the measurement object, in particular the measuring position, and the sensor. In other words, the traction cable represents a mechanical connection, in particular a kinematic connection between the measurement object, in particular the measuring position and the sensor. Preferably, the traction cable is mechanically connected to the measurement object. Preferably, the traction cable is mechanically connected to the sensor. A location change or position change of the measuring position can preferably cause an equal change in the length or position of the sensor on the sensor tape (or a change with a defined step-up) via the traction cable. The mechanical properties of the cable can depend on different environmental conditions and/or parameters, such as, for example, radiation intensity, magnetic fields, application of force, for example by a (return) spring, temperature and humidity. The properties of the traction cable, such as, for example, radiation resistance, magnetic properties, technical elongation, temperature resistance and corrosion properties can preferably be designed according to the environmental conditions and/or parameters present when ascertaining position information for a measuring position.

The deflection pulley can preferably change the direction of the traction cable in a targeted manner. Preferably, the deflection pulley can deflect the traction cable from a first direction, which runs between the measuring position of the deflection pulley, into a second direction, which runs between the deflection pulley and the sensor. In other words, in particular an alignment angle between the first direction and the second direction can be defined via a deflection pulley. Preferably, the traction cable can be guided by the deflection pulley. In particular, the deflection pulley can have a groove, indentation or track embodied to receive the traction cable and to guide the traction cable.

The measuring system advantageously enables robust and exact measurement over the entire measuring range similar to those of a linear measuring system. A particularly advantageous feature is the ability to spatially separate the measuring system and the measurement object. This enables position measurement on measuring objects in a way that is not possible with conventional measuring systems, for example linear measuring systems.

In one possible embodiment of the measuring system, a position change of the measuring position of the measurement object in the first direction is measured via the sensor by a position change of a position of the sensor on the sensor tape in the second direction.

A location change or position change of the measuring position (in a first direction) can preferably cause an identical position change (or a position change with a defined step-down/step-up) of the sensor on the sensor tape (in a second direction) via the traction cable. Preferably, a vertical position change of the measuring position of a measurement object can be determined by a horizontal displacement of the sensor on the sensor tape. The second direction is preferably horizontal, but can also have an inclination. The first direction is preferably vertical, but can also have an inclination. Preferably, the first direction and the second direction are orthogonal to one another. The first direction of the position change of a measuring position in particular refers to the direction in which movement or displacement of the measuring position takes place. The second direction of the position change of a sensor in particular refers to the direction in which movement or displacement of the sensor takes place. Preferably, the first and the second direction can be determined in a coordinate system. The coordinate system can in particular correspond to the coordinate system that is used to change the position of a measuring position. The coordinate system can in particular be arranged at a point, for example the starting point, of the measuring system, in particular the sensor tape. The second direction is preferably invariable and/or constant over a period of time, in particular a measurement duration. The second direction is preferably also constant over a period of time, in particular a measurement duration, but can also be non-constant. In other words, the first direction (of the measuring position) can vary from a constant second direction over a period of time, in particular a measurement duration.

The measuring system advantageously enables the measurement of a position and/or distance in a first (variable) direction via a measurement in a second (defined) direction. This enables flexibility with regard to the arrangement and measuring method of the measuring system, since the measurement always takes place in a defined measuring direction. In addition, a space-saving and compact design is possible, so that the construction costs of the apparatus can be reduced.

In one possible embodiment of the measuring system, the first direction extends vertically and the second direction extends horizontally.

Preferably, the movement of the measuring position takes place on a vertical or perpendicular axis and/or direction. Preferably, the movement of the sensor on the sensor tape extends horizontally and/or parallel to the floor. Preferably, the first direction and second direction are therefore arranged orthogonal to one another. In other words, the angle between the directional axes of the first and second direction is 90°. The traction cable preferably also extends perpendicularly in the first direction and horizontally in the second direction.

The measuring system advantageously enables the measurement of a vertical position change based on a horizontal measurement. Depending on the measuring method, a horizontal measurement can be more advantageous than a vertical measurement, since, for example, a vertical measurement can be influenced by the gravitational force of the Earth. For example, a horizontal measurement of vertical position changes by the measuring system can enable a large vertical measuring range.

Alternatively, in one possible embodiment, the first direction can extend horizontally and the second direction can extend vertically. For example, the movement of the sensor on the sensor tape can extend parallel to a wall arranged vertically (to a horizontal floor). Therefore, this can advantageously also enable vertical measurement of a horizontal position change. Advantageously, for example, the gravitational force can be used as a restoring force for the sensor. In particular, the measuring system can enable the measurement of a large horizontal measuring range.

In one possible embodiment of the measuring system, the traction cable is mechanically connected to the measurement object and/or the sensor.

The mechanical connection, preferably between the measurement object and the sensor via the traction cable, leads in particular to a corresponding displacement of the sensor on the sensor tape on a position change of the measuring position. In other words, in particular a movement of the measurement object can be measured by the measuring system, in particular the sensor. Alternatively, the traction cable can in particular establish a mechanical connection between a deflection pulley arranged on the sensor and the measuring position. The connection or attachment to the respective components, in particular the sensor and the measurement object, is preferably established such that it is possible to replace the traction cable, while at the same time ensuring a high level of safety. The traction cable in particular provides a direct connection between the measuring position and the position of the sensor. In this case, the traction cable is preferably constantly under tensile stress. This ensures error-free deflection of the traction cable and a direct transfer of the measuring position to the sensor. Alternatively, spindles or other connecting elements can be used instead of a cable.

Advantageously, a mechanical connection between the measurement object and sensor via the traction cable enables direct transmission of the position and/or position change of the measuring position to the sensor. This enables an exact measurement of the measuring position at a point on the sensor tape (sensor measuring point) that is at a distance from the measuring position.

In one possible embodiment of the measuring system, the measuring system comprises a guide. The measuring system guide guides the sensor on the sensor tape.

Preferably, the guide is embodied only to allow the sensor to move in one direction, for example in the x-direction, in a coordinate system arranged in a starting point. In particular, sideways and/or lateral movement and/or rotation and/or tilting of the sensor can be restricted by the guide. The guide can preferably comprise a rail and a carriage receiving the sensor. The guide can alternatively in particular be part of the sensor tape. The guide can be arranged in particular in/on a housing, which can in particular comprise the sensor and/or the sensor tape.

The guide can advantageously ensure exact positioning of the sensor on the sensor tape and therefore reduce measuring inaccuracies and/or enable precise measurement of the measuring position.

In one possible embodiment of the measuring system, the measuring system comprises a return spring. The return spring is preferably mechanically connected to the sensor and/or traction cable.

Preferably, the return spring enables exact positioning of the sensor on the sensor tape. The return spring can preferably be arranged between the sensor and a non-movable attachment point of the measuring system. The non-movable attachment point can in particular be arranged on an end of the sensor tape. Preferably, the non-movable attachment point can be arranged on a housing surrounding the measuring system. In addition, the return spring can in particular be arranged between the traction cable and a non-movable attachment point. At the same time, the return spring can exert a tensile force on the traction cable and/or the sensor. Alternatively, the return spring can also exert a compressive force on the sensor and/or the traction cable. In particular, a tensile or compressive force generated by the return spring can be constant. Furthermore, the return spring can be embodied to transmit an increasing or decreasing force, in particular a linear force, to the sensor and/or the traction cable. For example, the compressive force of the return spring can be maximum in an initial state and minimum in a maximum measuring state.

In addition, the measuring system can comprise more than one (return) spring. For example, the measuring system can have a spring between the sensor and the non-movable attachment point and a further spring between the traction cable and the non-movable attachment point and/or sensor. For example, two or more springs can be arranged in series, in order in particular to transmit a specific spring force and/or restoring force to the traction cable. Furthermore, different springs and/or spring types can be combined and/arranged together to form a spring system, in particular return spring system. In particular, a defined spring characteristic curve can be determined via such a spring system, in particular return spring system. For example, the arrangement of a kinematic deflection element on the spring is conceivable, in particular in order to exert a constant tensile force, in particular restoring force, on the traction cable. Advantageously, the return spring can, for example, comprise a gas spring. A gas spring can preferably provide a constant restoring force. Furthermore, the return spring can comprise a spring system, in particular a return spring system. A spring system, in particular a return spring system, can in particular comprise a spring balancer. A spring balancer can in particular comprise a torsion spring in conjunction with a constantly variable cable radius of a take-up spool, in particular to provide a constant restoring force and/or tensile force. Instead of a return spring, it is also possible to use another object with a comparable function. For example, a foam or an object made of compressible material can be used.

A return spring can advantageously enable guidance and/or positioning of the sensor on/at the sensor tape and increase the measuring accuracy of the measuring system.

In one possible embodiment of the measuring system, the measuring system comprises a return spring. The tension spring can apply a restoring force to the traction cable and/or the sensor.

Preferably, the sensor and the traction cable are loaded via a return spring. Applying a restoring force to the sensor can in particular ensure that the sensor returns to a measuring starting point upon a corresponding change in the measuring position of the measurement object. The application of a restoring force to the traction cable can in particular ensure a constant tensile stress state of the traction cable. This can in particular enable error-free guidance and/or deflection of the traction cable by the deflection pulleys. In other words, a constantly applied tension ensures the traction cable is in a state in which no knotting, entanglement or sagging of the traction cable can occur.

The application of a restoring force to the sensor and/or the traction cable can advantageously ensure the direct transmission of the measuring position of the measurement object to the sensor measuring position on the sensor tape.

In one possible embodiment of the measuring system, the measuring system comprises at least one further deflection pulley. The deflection pulley deflects the traction cable.

A further deflection pulley can in particular enable precise alignment and/or guidance of the traction cable. Preferably, the at least one further deflection pulley is arranged at a distance to a first deflection pulley. Due to the geometric properties of the measurement object, a plurality of deflections of the traction cable may be necessary, for example, in order to transmit the measuring position of the measurement object to the sensor tape. For example, the traction cable can be guided around an obstacle (object) located between a first deflection pulley and the measuring position. In addition, in particular a step-down/step-up of the traction cable can be enabled by a further deflection pulley. Preferably, however, in this case, the measuring system in this case is designed with as few deflection pulleys as possible. A deflection pulley can also be used to stabilize or guide the traction cable and can therefore be referred to differently, for example as a pulley or a guide pulley.

Advantageously, further deflection pulleys can improve the alignment or guidance of the traction cable. This can in particular lead to improved transmission of the (vertical) measuring position to the sensor tape of the measuring system.

In one possible embodiment of the measuring system, the measuring system comprises at least one further deflection pulley that is mechanically connected to the sensor, in particular rigidly.

In order in particular to enable a step-down/step-up, the deflection pulley can be arranged on the sensor. The mechanical connection between sensor and deflection pulley can in this case in particular be rigid. Alternatively, the mechanical connection can for example be established by an elastic element. Preferably, the sensor is attached to a frame element and/or housing element of the sensor. In other words, the deflection pulley can also be attached to the sensor. Attaching the deflection pulley to the sensor enables a deflection of the traction cable on the sensor. The deflection of the traction cable on the sensor enables a step-down/step- up of the measuring system similar in function to a block and tackle. In particular one or more further deflection pulleys can be attached to the sensor.

Advantageously, deflection of the traction cable on the sensor enables a particularly compact design of the measuring system.

In one possible embodiment of the measuring system, the measuring system comprises a non-movable attachment point to which the traction cable is attached.

The non-movable attachment point can in particular be arranged on an end of the sensor tape. Preferably, the non-movable attachment point can be arranged on a housing surrounding the measuring system. For example, the attachment point can also be arranged on a deflection pulley. In particular, the attachment of the traction cable to a non-movable attachment point of the measuring system can enable a step-down of the traction cable. The attachment point is in particular non-movable with respect to the sensor tape or a housing (surrounding the measuring system). The traction cable can in particular be fastened by a (cable) clamp, hooks, eyelets, split pins or screws. In particular, the attachment can be effected in such a way that the replacement, adjustment or re-tensioning of the traction cable is possible.

Advantageously, attaching the traction cable to a non-movable attachment point enables simplified attachment of the traction cable, in particular since the traction cable is not attached to the sensor. This can advantageously lead to improve guidance and/or positioning of the sensor on the sensor tape.

In one possible embodiment of the measuring system, the measuring distance ascertained by the sensor on the sensor tape corresponds to an integer multiple of the position distance of the measuring position.

The deflection pulleys of the measuring system can preferably act like a block and tackle with/by a corresponding factor, depending on the arrangement of the deflection pulleys. In other words, the arrangement of the deflection pulleys in the measuring system can enable a step-down/step-up. In particular, a measuring distance and/or necessary restoring force can be reduced by a step-down/step-up. In particular, the factor can be an integer number, for example 1, 2 or 5. A correspondingly selected factor in particular enables a compact design of the measuring system and/or a short sensor tape. In particular, a step-up of the measuring distance of the measuring position into a measuring distance of the sensor on the sensor tape can be applied with a factor for large or long (vertical) measuring distances.

A step-down advantageously enables scaling of the measuring system. In particular, large vertical measuring distances can be measured with a short sensor tape and/or a compact measuring system.

In one possible embodiment of the measuring system, the measuring system comprises a housing.

In particular, the housing can comprise the sensor and the sensor tape. Preferably, the housing can also comprise one or more deflection pulleys. Preferably, the housing can also comprise one or more (return) springs. In other words, the measuring system can be housed by a protective component. Preferably, the housing has an opening through which the traction cable can enter and/or exit the housing. In particular, the opening of the housing can have a guide and/or seal. In particular, the seal can be embodied to prevent and/or reduce the ingress of dirt and/or dust particles into the housing.

In particular, the housing can advantageously prevent and/or reduce soiling of the sensor and/or the sensor tape. Mechanical components, such as, for example, a guide holding the sensor, can thus be protected against wear. Enclosed measuring systems and/or housed measuring systems can be less sensitive to external influences, such as dust particles, and enable a more reliable measurement due to a reduced selection probability.

One possible embodiment of the present invention comprises a table comprising a lifting device, further comprising the measuring system (in one of the possible embodiments). The height of the table can be adjusted via the lifting device. The height of the table can be determined via the measuring system. The lifting device of the table can in particular comprise a scissor lifting device.

For example, the table can be a patient bench or a patient table. In particular, the table can be embodied to accommodate a patient. Preferably, the table can be a component of a medical apparatus. In particular, the table can be raised to a precise vertical height. The measuring system enables precise determination of the height of the table. In the case of a scissor lift table, for example, it may not be possible to attach conventional linear measuring systems to the movement axes. Furthermore, the measuring system can preferably enable the lifting speed to be measured and/or determined during a movement of the table.

Advantageously, the measuring system enables the height of the table to be measured precisely. In this case, the measuring system is advantageously an apparatus that does not have to be arranged directly on the movement axis or lifting axis. The specific and/or measured lifting speed can advantageously additionally enable the control of a drive of the table.

One possible embodiment of the present invention comprises a medical apparatus comprising the table and/or the measuring system. The medical apparatus is in particular embodied to record medical image data.

In particular, the medical apparatus can be a medical imaging system. A medical imaging system can generally be embodied to generate medical image data. Medical image data can generally be image data of a part of a patient's body. Accordingly, medical imaging systems are embodied to map patients' body parts. In particular, medical imaging systems can implement radiological imaging methods. Medical imaging systems can comprise one or more imaging modalities such as, for example, computed tomography devices, magnetic resonance devices, X-ray devices or ultrasound devices and the like. Alternatively, the medical system can be a therapy apparatus. In particular, the medical system can be an irradiation facility by which a patient arranged on a (patient) table can be treated with radiation in a locally defined body region.

In particular in the case of medical apparatuses, in particular medical imaging methods, local separation of the measuring system from the measuring position is particularly advantageous. The radiation (or magnetic fields) for imaging and/or therapy can influence and/or impair the electronics in measuring systems. Coupling the measuring position to the sensor of the measuring system via a traction cable enables influences on the measuring system, for example by a magnetic field of a magnetic resonance apparatus, to be largely avoided by spatial separation.

### BRIEF DESCRIPTION OF THE DRAWINGS

Further advantages, features and details of the present invention emerge from the exemplary embodiments described below and from the drawings. Corresponding parts are provided with the same reference symbols in all figures. There is no repeated description of corresponding parts in the respective exemplary embodiments. Exemplary embodiments can substantially differ in the arrangement of the units.

The figures show:

FIG. 1 a measuring system for measuring the position of a measurement object that can be moved in a first direction

FIG. 2 a measuring system for measuring the

position of a measurement object that can be moved in a first direction with step-down

FIG. 3 a (scissor lift table) table with a measuring system for height measurement in the initial state

FIG. 4 a (scissor lift table) table with a measuring system for height measurement in a measuring position.

### DETAILED DESCRIPTION

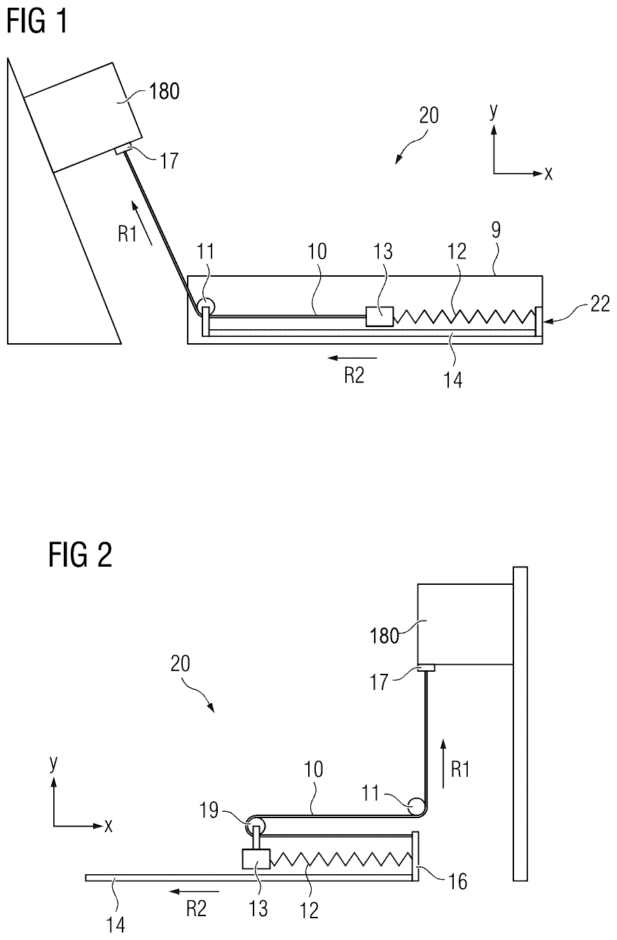

FIG. 1 is a schematic representation of a measuring system 20 suitable for measuring the position of a measurement object 180 that can be displaced in a first direction R1. A traction cable 10 attached to a measuring position 17 transmits the position or movement (position change) of the measurement object 180 (or the measuring position 17) to the sensor 13. The sensor 13 moves in a direction R2 on a sensor tape 14 and is connected to the traction cable 10. The deflection pulley 11 is arranged between the measuring position 17 and sensor 13 and deflects the traction cable 10 from a direction R1 into a direction R2. Positioning the deflection pulley 11 at the end of the sensor tape 14 enables the traction cable 10 to be guided parallel and/or horizontally to the sensor tape 14. A return spring 12 is attached to the sensor 13. In addition, the return spring 12 is attached to an end of the sensor tape 14 and/or to a housing 9. The return spring 12 returns the sensor 13 to its initial state when the measurement object 180 moves against the measuring direction R1. In addition, the return spring 12 ensures that the traction cable 10 is always under tension. Contrary to what is shown, a second spring can also be provided, for example between a starting point 22 and the traction cable 10. The measuring system 20 can be surrounded by a housing 9. In particular, as shown, the housing 9 can comprise the sensor 13, the sensor tape 14, a deflection pulley 11 and the return spring 12. Contrary to what is shown, the housing 9 can also comprise further deflection pulleys. Likewise, contrary to what is shown, the housing 9 may not comprise components of the measuring system 20, in particular the deflection pulley 11. The traction cable 10 can enter and exit the housing through an opening. A guide or a seal can be provided at the opening of the housing 9 in order to enable the traction cable 10 to be guided as friction-free as possible without dirt particles being able to enter the housing.

FIG. 2 is a schematic representation of a measuring system 20 suitable for measuring the position of a measurement object 180 that can be displaced in a first direction R1 with step-down. The return spring 12 and the traction cable 10 are attached to an attachment point 16 of the measuring system. The return spring 12 is connected to the sensor 13. A further deflection pulley 19 is attached to the sensor 13. The deflection pulley 19, which is also referred to as a loose pulley, guides the traction cable 10 from the attachment point 16 to the deflection pulley 11. The deflection by 180° causes the traction cable to initially run in the direction R2 and then in the opposite direction after the deflection. Directions R1 and R2 are orthogonal to one another, direction R1 perpendicular/vertical and direction R2 horizontal. In this exemplary embodiment, the reduction factor is 2. A position change of the measuring position 17 in direction R1 results in half as large a displacement of the sensor 13 in direction R2.

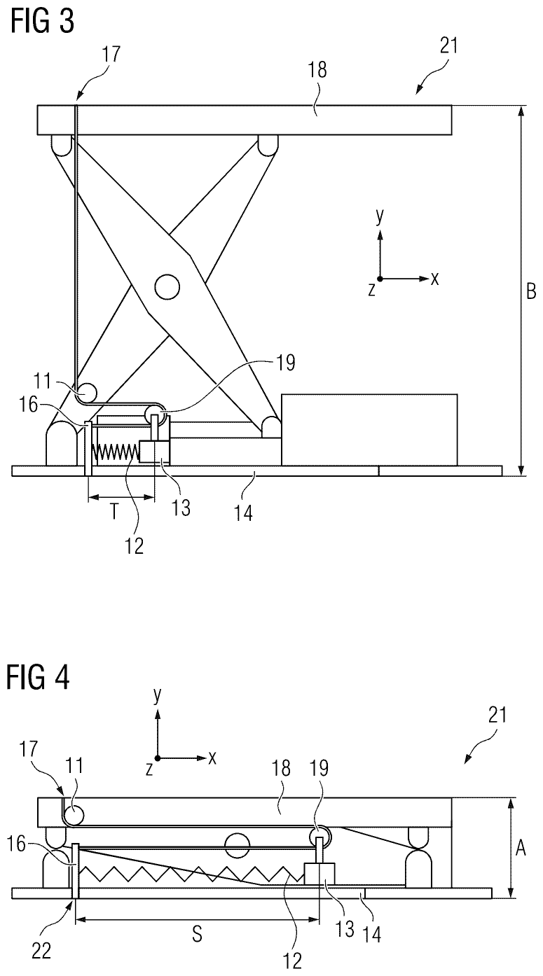

FIG. 3 and FIG. 4 show an apparatus (scissor lift table) 21 consisting of a table segment 18 with scissor lift and an (integrated) measuring system consisting of a sensor 13, a sensor tape 14, a return spring 12, two deflection pulleys 11, 19 for height measurement from one side. In this case, the measurement object is the table segment 18 and/or the surface of the table segment 18 of the scissor lift table 21.

FIG. 3 shows the scissor lift table 21 in its initial state or retracted state. In this case, initial state typically refers to the state in which the table segment 18 has the lowest height A (in the y-direction of the coordinate system). The height A indicates the distance between the surface of the table segment 18 and the floor on which the scissor lift table 21 is positioned. The sensor tape 14 and the sensor 13 (and the return spring 12 and the deflection pulley 19) of the measuring system are arranged horizontally in the lower region of the scissor lift table near the floor. In the initial state of the scissor lift table, the sensor 13 is in a maximally deflected position on the sensor tape. The maximally deflected position of the sensor 13 on the sensor tape 14 corresponds to a distance S between the end of the sensor tape 14, to which a return spring and/or the traction cable (attachment point 7 is attached, and the sensor 13. In other words, in the initial state, the distance A characterizing the measuring distance corresponds to a distance S characterizing the measuring position on the sensor tape. The minimum value of the height (distance A) corresponds to the maximum value of the sensor distance (distance S).

FIG. 4 shows the state in which the scissor lift table 21 is extended to its maximum extent. In this maximum state, the maximum value of the height of the table segment 18 to the floor, distance B, is reached. The maximum value of the height (distance B) corresponds to the minimum value of the sensor distance (distance T). When the scissor lift table is extended, the measuring distance (the height of the table) increases with a simultaneous change to the sensor distance (positioning of the sensor on the sensor tape compared to the initial state).

The scissor lift kinematics can be embodied as single scissors, as shown in FIG. 3 and FIG. 4, or as double or multiple scissors. Other lifting kinematics are conceivable that have a linear upward lifting movement. In addition to the position of the lifting table, the proposed measuring system can also enable the speed of the scissor lift table to be measured or determined.

Finally, reference is made once again to the fact that the figures of the measuring system shown are merely exemplary embodiments which can be modified by the person skilled in the art in a wide variety of ways without departing from the scope of the present invention. For example, (contrary to what is shown) features of different exemplary embodiments can, for example, be combined. Furthermore, the use of the indefinite articles “a” or “an” does not exclude the possibility that the features in question may be present more than once. Likewise, the term “system” does not exclude the possibility that the components in question consist of a plurality of interacting sub-components, which may also be spatially distributed.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A measuring system for ascertaining position information for a measuring position of a measurement object, the measuring system comprising:
a sensor tape;
a sensor configured to capture position information on the sensor tape;
a traction cable configured to transmit the measuring position of the measurement object to the sensor; and
at least one deflection pulley configured to deflect the traction cable from a first direction to a second direction.

2. The measuring system as claimed in claim 1, wherein the measuring system is configured to measure a position change of the measuring position of the measurement object in the first direction via the sensor by a position change of a position of the sensor on the sensor tape in the second direction.

3. The measuring system as claimed in claim 1, wherein the first direction is a vertical direction and the second direction is a horizontal direction.

4. The measuring system as claimed in claim 1, wherein the traction cable is mechanically connected to at least one of the measurement object or the sensor.

5. The measuring system as claimed in claim 1, further comprising:
a guide configured to guide the sensor on the sensor tape.

6. The measuring system as claimed in claim 1, further comprising:
a return spring that is mechanically connected to the sensor and the traction cable.

7. The measuring system as claimed in claim 1, further comprising:
a return spring configured to apply a restoring force to at least one of the traction cable or the sensor.

8. The measuring system as claimed in claim 1, further comprising:
at least one further deflection pulley configured to deflect the traction cable.

9. The measuring system as claimed in claim 8, wherein at least one deflection pulley is mechanically connected to the sensor.

10. The measuring system as claimed in claim 1, wherein the traction cable is attached to a non-movable attachment point of the measuring system.

11. The measuring system as claimed in claim 1, wherein the position information captured by the sensor corresponds to an integer multiple of a position distance of the measuring position.

12. A table comprising:
the measuring system as claimed in claim 1; and
a lifting device by which a height of the table is adjustable in the first direction, wherein
the measuring system is configured to determine the height of the table.

13. A medical apparatus comprising:
the table as claimed in claim 12.

14. The measuring system as claimed in claim 2, wherein the first direction is a vertical direction and a second direction is a horizontal direction.

15. The measuring system as claimed in claim 2, wherein the traction cable is mechanically connected to at least one of the measurement object or the sensor.

16. The measuring system as claimed in claim 2, further comprising:
a guide configured to guide the sensor on the sensor tape.

17. The measuring system as claimed in claim 2, further comprising:
a return spring that is mechanically connected to the sensor and the traction cable.

18. The measuring system as claimed in claim 17, wherein the return spring is configured to apply a restoring force to at least one of the traction cable or the sensor.

19. The table of claim 12, wherein the lifting device is a scissor lifting device.

20. A medical apparatus comprising:
a table; and
the measuring system as claimed in claim 1.
